(19) United States
(12) Patent Application Publication
(10) Pub. No.: US 2013/0180527 A1
(43) Pub. Date: Jul. 18, 2013

(54) BAG VALVE MASK FOR APPROPRIATE VENTILATION

(71) Applicants: Do-Hee Kim, Daejeon (KR); Seung-Whan Kim, Daejeon (KR); O-Yu Kwon, Daejeon (KR)

(72) Inventors: Do-Hee Kim, Daejeon (KR); Seung-Whan Kim, Daejeon (KR); O-Yu Kwon, Daejeon (KR)

(21) Appl. No.: 13/876,210

(22) PCT Filed: Oct. 7, 2010
(86) PCT No.: PCT/KR2010/006874

§ 371 Date: Mar. 27, 2013

(30) Foreign Application Priority Data
Sep. 27, 2010 (KR) 10-2010-0093317

(51) Int. Cl. A61M 16/00; A61M 16/08; A61M 16/20; A61M 16/06

(52) U.S. Cl. CPC A61M 16/0078; A61M 16/06; A61M 16/0875; A61M 16/20; A61M 16/0051
USPC 128/205.13

(57) ABSTRACT

According to the present invention, a bag valve mask (BVM) for appropriate ventilation, comprising a pressurization portion formed at an air bag, wherein the pressurization portion is the pressurization point pressurized by tips of a user fingers while encompassing the air bag with the fingers, to continuously ventilate a patient with a predetermined amount of air using artificial respiration. More specifically, the BVM comprises: a mask closely adhered to a patient's nose and mouth so as to be isolated from the outside; an air bag in which oxygen or air is accommodated; and a connection hose for communicating between the mask and the air bag. The air bag is configured in such a way that the accommodated oxygen or air flows through to a patient's trachea via the connection hose and the mask, and an identification means is formed at the air bag to identify the pressurization portion.

### TECHNICAL FIELD

The present invention relates to a bag valve mask (BVM) for appropriate ventilation, and more particularly, to a BVM for ventilating a patient with an appropriate, constant ventilation amount of air (oxygen): 500 cc to 600 cc.

### BACKGROUND ART

Generally, BVMs are emergency medical devices widely used in 119 ambulances (911 ambulances in some countries), hospital ambulances, emergency centers, intensive care units, operating rooms, recovery rooms, general wards, etc. for patients undergoing cardiopulmonary dysfunction or arrest.

A BVM may be effectively used for resuscitating a patient in the following manner. While continuously pushing an airbag of the BVM with one hand, a rescuer treats a patient with his other hand and monitors other symptoms of the patient or other dangerous situations with his eyes.

Guidelines for cardiopulmonary resuscitation published in 2005 by American Heart Association and European Society of Cardiology have recommended manual resuscitators (bag valve masks, BVMs) having ventilation capability of 500 to 600 cc at a time. However, since BVMs usually have ventilation capability of about 1500 to 1200 cc (BVMs produced in Korea usually have ventilation capability of about 1700 cc), it is difficult to keep the amount of ventilation constant using BVMs.

The amount of ventilation to a patient is largely varied according to the skill, competence, or ability of an operator, and this has been proved by various experimental data and papers.

However, as far as the inventors know, a BVM indicating the amount of ventilation has not yet been introduced or commercialized.

To display the flow rate of air, a flow meter may be attached to an adaptor through which air is supplied to a patient. However, it may be difficult to frequently check the flow meter in emergency situations. In addition, installation of a special device or complicated equipment may also be not preferable.

### SUMMARY OF INVENTION

### Technical Problem

To solve the above-mentioned limitations, the present invention provides a bag valve mask (BVM) for ventilating a patient with an appropriate, constant ventilation amount of air (oxygen): 500 cc to 600 cc.

### Solution to Problem

The present invention has been made in an effort to solve the above problems and is directed to a bag valve mask (BVM) for appropriate ventilation, the BVM including pressurization portions formed on predetermined positions of an air bag as pressurization points which are pressed by tips of fingers of a rescuer when the rescuer holds the air bag with his fingers to artificially respirate a patient by continuously ventilating the patient with a constant amount of air.

In detail, the BVM may include: a mask configured to make contact with the noise and mouth of the patient in a state not allowing inflow of ambient air; the air bag containing oxygen or air; and a connection hose connecting the mask and the air bag, wherein if the air bag is compressed, the oxygen or air contained in the air bag may be supplied to the patient through the connection hose, the mask, and the respiratory tact of the patient, wherein identifiers may be formed on the air bag to indicate the pressurization portions.

The identifiers may be boundary lines printed as identification marks around regions of the air bag which are brought into contact with the tips of the fingers of the rescuer when the air bag is compressed by the rescuer.

One of the boundary lines may be printed on an outer side of the air bag at a position spaced 3 cm in a vertical direction from a center point on a longitudinal mid-line of the air bag so that a thumb of the rescuer can be placed thereon, and another of the boundary lines may be printed on the other outer side of the air bag at a position spaced 3 cm in the vertical direction from the center point so that a middle finger of the rescuer can be placed thereon, wherein other of the boundary lines may be printed in a manner such that an index finger of the rescuer is located in front of the middle finger, and a ring finger and a little finger of the rescuer are located in back of the middle finger in a longitudinal direction of the air bag.

Alternatively, one of the boundary lines may be printed on the outer side of the air bag at a position spaced 3 cm in the other vertical direction from the center point on the longitudinal mid-line of the air bag so as to indicate a thumb position, and another of the boundary lines may be printed on the other outer side of the air bag at a position spaced 3 cm in the other vertical direction from the center point so as to indicate a middle finger position, wherein other of the boundary lines may be printed so as to indicate an index finger position in front of the middle finger, and a ring finger position and a little finger position in back of the middle finger position along the longitudinal direction of the air bag.

Alternatively, boundary lines may be printed on the outer side of the air bag at positions spaced 5 cm in both vertical directions from the center point on the longitudinal mid-line of the air bag so as to indicate thumb positions, respectively, and boundary lines may be printed on the other outer side of the air bag at positions spaced 5 cm in both the vertical directions from the center point so as to indicate middle finger positions, wherein boundary lines may be printed along the longitudinal direction of the air bag so as to indicate index finger positions in front of the positions for the middle finger positions, and ring finger positions and little finger positions in back of the middle finger positions.

The boundary lines may be rectangles having a side length in a range from 1.9 cm to 2.1 cm.

Alternatively, the boundary lines may be circles having a radius in a range from 0.9 cm to 1.1 cm.

Concave identification recesses may be formed inside the boundary lines.

The identification recesses may be formed as a thumb identification recess and a middle finger identification recess on insides of the boundary lines on which a thumb and a middle finger of the rescuer are to be placed, wherein when the air bag is compressed, inner surfaces of the air bag may be brought into contact with each other at the thumb and middle finger identification recesses.

The identifiers may be concave identification recesses formed as identification shapes at positions of the air bag which make contact with the fingers of the rescuer when the air bag is compressed by the rescuer.

The identifiers may be an identification color(s) painted as identification marks on positions of the air bag which make contact with the fingers of the rescuer when the air bag is compressed by the rescuer.

The identifiers may include: concave identification recesses formed at positions of the air bag which make contact with the fingers of the rescuer when the air bag is compressed by the rescuer; and an identification color(s) painted on the identification recesses.

The pressurization portions may be formed at the predetermined positions of the air bag so that the patient is ventilated with 500 to 600 cc of oxygen or air when the air bag is compressed at the pressurization portions.

In other embodiments, the pressurization portions may be formed at the predetermined positions of the air bag so that the patient is ventilated with 400 to 500 cc of oxygen or air when the air bag is compressed at the pressurization portions.

The BVM of the present invention may further include an oxygen or air cylinder disposed on the air bag.

The air bag may be formed of silicone or rubber.

The connection hose may include a one-way valve allowing oxygen or air to flow only in one direction from the air bag to the mask.

An alarming unit may be disposed on the air bag or the connection hose to provide information about time.

The alarming unit may produce a basic sound once per second and a discriminating sound distinguishable from the basic sound every six seconds.

### Advantageous Effects of Invention

According to the present invention, in the BVM for appropriate ventilation, the pressurization portions are formed on predetermined positions of the air bag as pressurization points so that a rescuer can continuously ventilate a patient with a constant amount of air during artificial respiration by holding the air bag with his fingers and pressing the pressurization portions of the air bag with his finger tips. Therefore, patients can be ventilated with a constant, appropriate ventilation amount of air: 500 to 600 cc by using the BVM.

Since an approximate ventilation amount of air (oxygen) can be supplied precisely and continuously by using the BVM, patients undergoing a cardiac arrest or cardiovascular collapse can be properly treated for recovery.

### BRIEF DESCRIPTION OF DRAWINGS

FIG. 1 is a perspective view illustrating a bag valve mask (BVM) for appropriate ventilation according to an exemplary embodiment of the present invention.

FIG. 2 is a schematic view illustrating the BVM for appropriate ventilation of FIG. 1.

FIG. 3 is a view illustrating the BVM for appropriate ventilation of FIG. 1 when the BVM has circular boundary lines.

FIG. 4 is a view illustrating the BVM for appropriate ventilation of FIG. 1 when the BVM has colorful identifiers.

FIG. 5 is a view illustrating the BVM for appropriate ventilation of FIG. 1 when the BVM is configured for left-handers.

FIG. 6 is a view illustrating the BVM for appropriate ventilation of FIG. 1 when the BVM is configured for ambidexters.

FIG. 7 is a view illustrating the BVM for appropriate ventilation of FIG. 1 when the BVM has identification recesses as identifiers.

FIG. 8 is a view illustrating the BVM for appropriate ventilation of FIG. 7 when the BVM is compressed by the fingers of a rescuer.

FIG. 9 is a graph showing the ventilation amounts of a BVM of the related art and the ventilation amounts of the BVM of FIG. 1 while the BVMs are used a plurality of times.

### DESCRIPTION OF EMBODIMENTS

A bag valve mask (BVM) for appropriate ventilation of the present invention is characterized by pressurization portions that can be pressed by a rescuer for ventilating a patient with an appropriate amount of oxygen or air.

The present invention will now be described in detail with reference to the accompanying drawings.

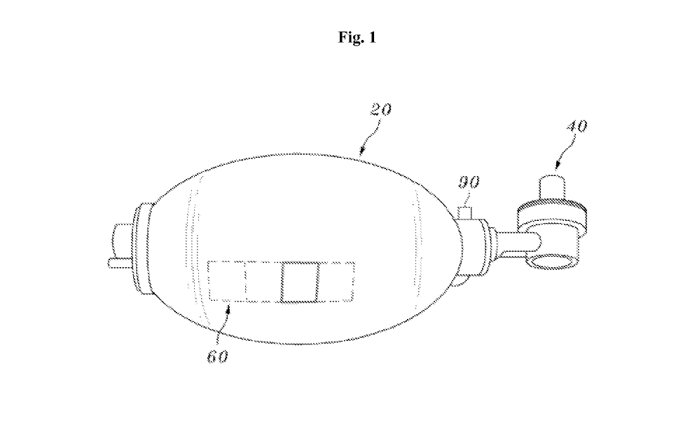

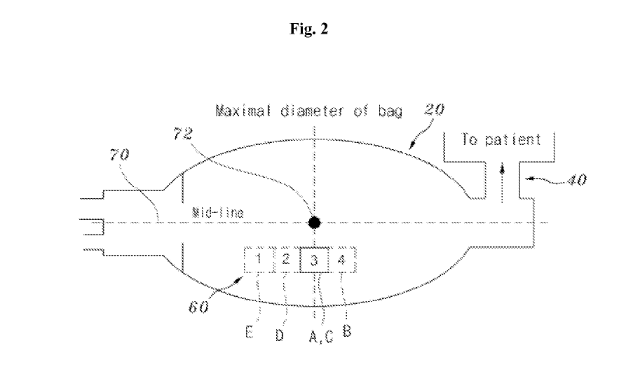

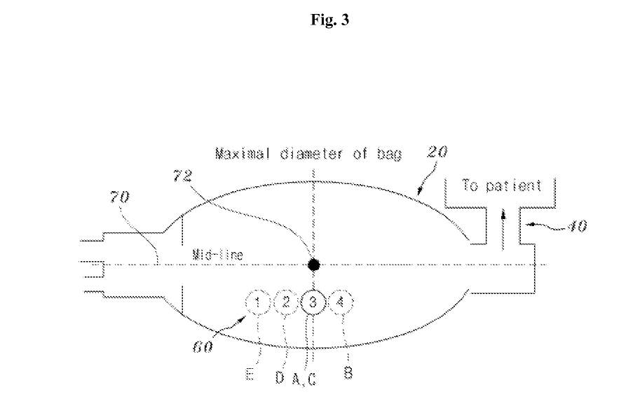

FIG. 1 is a perspective view illustrating a BVM for appropriate ventilation according to an exemplary embodiment of the present invention, FIG. 2 is a schematic view illustrating the BVM for appropriate ventilation of FIG. 1, and FIG. 3 is a view illustrating the BVM for appropriate ventilation of FIG. 1 when the BVM has circular boundary lines.

Referring to the drawings, the BVM of the present invention includes a mask, an air bag 20, and a connection hose 40 connecting the mask and the air bag 20.

The mask (not shown) is configured to make contact with the nose and mouth of a patient in a state not allowing inflow of ambient air.

The air bag 20 has an inner cavity to contain oxygen or air. The air bag 20 may be formed of silicone or rubber.

The connection hose 40 includes a one-way valve allowing oxygen or air to flow only in one direction from the air bag 20 to the mask.

The BVM of the present invention may further include an oxygen or air cylinder disposed at the air bag 20.

According to the present invention, if the air bag 20 is pressed, oxygen or air contained in the air bag 20 is supplied to a patient through the connection hose 40, the mask, and the respiratory tract of the patient.

To continuously ventilate a patient with a constant ventilation amount of air during artificial respiration, the air bag 20 of the present invention includes pressurization portions as pressurization points which are pressed by the tips of the fingers of a rescuer when the rescuer holds the air bag 20 with his hand and compresses the air bag 20.

Identifiers are formed on the pressurization portions.

The identifiers are identification marks formed by printing boundary lines 60 along the boundaries of regions which make contact with the finger tips of a rescuer when the rescuer presses the air bag 20.

A center point 72 is on a longitudinal mid-line 70 of the air bag 20, and one of the boundary lines 60 is printed on an outer side of the air bag 20 at a position spaced 3 cm in a vertical direction from the center point 72 so that the thumb (A) of a rescuer can be placed thereon.

Another of the boundary lines 60 is printed on the other outer side of the air bag 20 at a position spaced 3 cm in the vertical direction from center point 72 so that the middle finger (C) of the rescuer can be placed thereon.

The other of the boundary lines 60 are printed in a manner such that the index finger (B) of the rescuer can be located in front of the middle finger (C), and the ring finger (D) and little finger (E) of the rescuer can be located in back of the middle finger (C) in the longitudinal direction of the air bag 20.

The center point 72 is on a cross section of the air bag 20 having the largest diameter.

Then, if the BVM of the present invention is pressed, a patient can be ventilated with 500 to 600 cc of oxygen or air, and the ventilation air amount can be maintained constant.

In the present invention, the positions of the pressurization portions are not limited to the above-described positions. For example, other positions may be selected to ventilate a patient with 400 to 500 cc of oxygen or air. That is, the positions of the pressurization portions may be varied to supply an appropriate amount of oxygen or air according to patients.

Referring to FIGS. 2 to 6, a rescuer may hold the air bag 20 by placing his thumb (A) at a position 3 on an outer side of the air bag 20 and his middle finger (C) at another position 3 on the other outer side of the air bag 20.

At this time, the rescuer places his index finger (B) at a position 4, his ring finger (D) at a position 2, and his little finger (E) at a position 1 on the other outer side of the air bag 20.

Then, the rescuer may move his thumb (A) and middle finger (C) toward each other to push the air bag 20 and supply air to a patient.

As shown in FIGS. 2, 5, and 6, the boundary lines 60 may be rectangles having a side length in the range from 1.9 cm to 2.1 cm.

Alternatively, as shown in FIG. 3, the boundary lines 60 may be circles having a radius in the range from 0.9 cm to 1.1 cm.

Furthermore, the inner regions of the boundary lines 60 may be recessed to form identification recesses so that a rescuer can distinguish the pressurization portions from other regions by touching and hold the air bag 20 stably and comfortably. In addition, the identification recesses may prevent rescuer's fingers from slipping away from the pressurization portions.

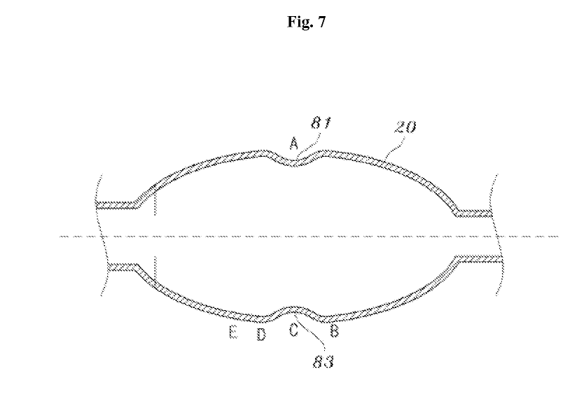

In detail, referring to FIG. 7, a thumb identification recess 81 is formed inside the boundary line 60 where the thumb (A) of a rescuer will be placed, and a middle finger identification recess 83 is formed inside the boundary line 60 where the middle finger (C) of the rescuer will be placed.

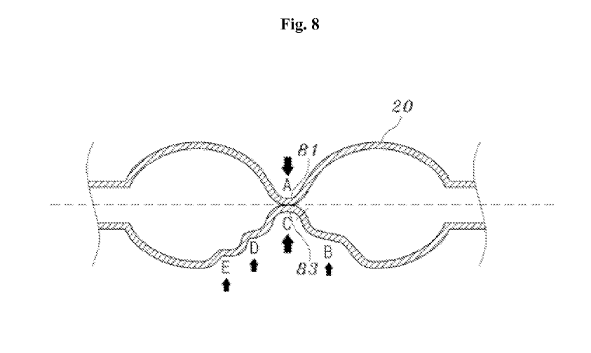

Therefore, when the air bag 20 is pressed, a portion of the thumb identification recess 81 is brought into contact with a portion of the middle finger identification recess 83 as shown in FIG. 8 so that a patient can be ventilated with an appropriate amount of air, for example, 500 to 600 cc of air. Owing to the middle finger identification recess 83 and the thumb identification recess 81, a rescuer may easily recognize the moment when the portions of the air bag 20 are brought into contact with each other, and thus the air bag 20 can be precisely pressed to ventilate a patient with a constant amount air.

Without forming the boundary lines 60, only the concave identification recesses may be formed at positions where the fingers of a rescuer will be placed when the rescuer presses the air bag 20.

Such identification recesses may be formed by attaching thimble-shaped moldings to the outside of the air bag 20 while pressing the moldings inwardly. However, the present invention is not limited thereto. That is, such identification recesses can be formed in various methods.

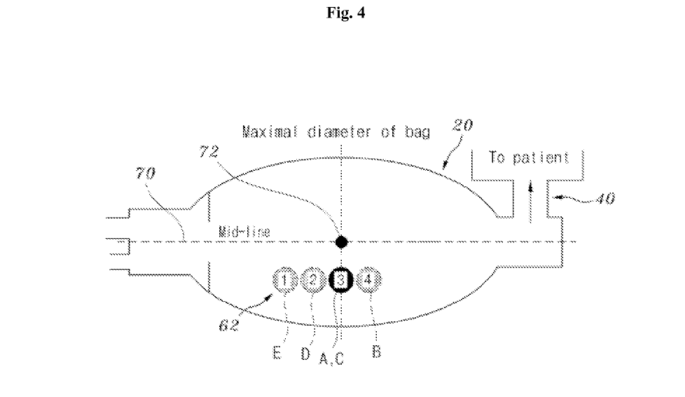

As shown in FIG. 4, the identifiers may be identification marks formed by painting an identification color(s) 62 on portions on which the fingers of a rescuer will be placed when the rescuer presses the air bag 20.

The identifiers may include: concave identification recesses formed on the portions of the air bag 20 on which the fingers of a rescuer will be placed when the air bag 20 is pressed; and an identification color(s) 62 painted on the recesses.

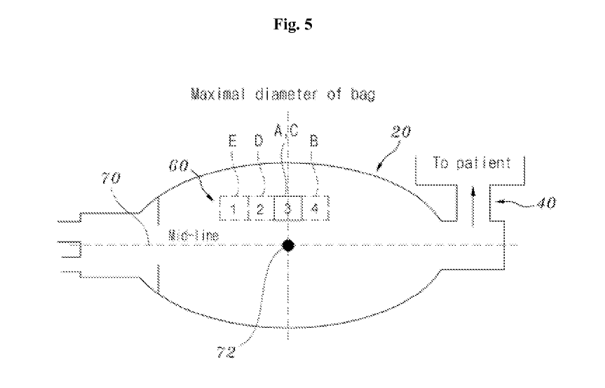

As well as the air bag 20 of the present invention being configured for right-handers, the air bag 20 can be configured for left-handers by changing the positions of the identifiers as shown in FIG. 5.

In detail, a boundary line 60 is printed on the outer side of the air bag 20 at a position spaced 3 cm in the other vertical direction from the center point 72 of the longitudinal mid-line 70 of the air bag 20, so as to indicate the position as a position for the thumb (A) of a rescuer. Another boundary line 60 is printed on the other outer side of the air bag 20 to indicate a position for the middle finger (C) of the rescuer.

In addition, other boundary lines 60 are printed along the longitudinal direction of the air bag 20, so as to indicate a position for the index finger (B) of the rescuer in front of the position for the middle finger (C), and positions for the ring finger (D) and little finger (E) of the rescuer in back of the position of the middle finger (C).

In other words, for right-handers, boundary lines 60 are printed on a vertical half of the air bag 20 based on the longitudinal mid-line 70 of the air bag 20, and for the left-handers, boundary lines 60 are printed on the other vertical half of the air bag 20.

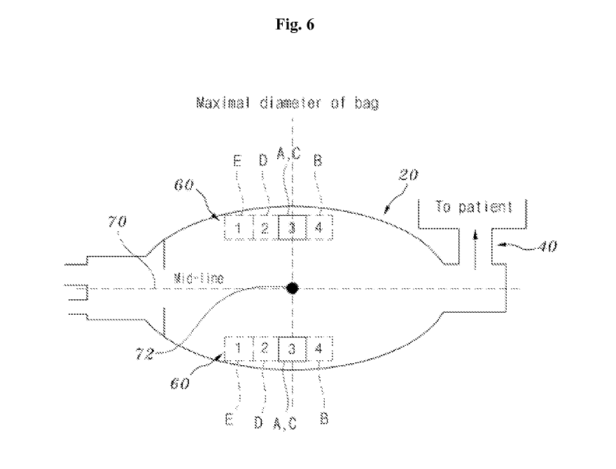

Furthermore, the air bag 20 of the present invention can be configured for ambidexters as shown in FIG. 6.

In detail, boundary lines 60 are printed on the outer side of the air bag 20 at positions spaced 5 cm in the vertical directions from the center point 72 of the longitudinal mid-line 70 of the air bag 20, so as to indicate positions for the thumb (A) of a rescuer. Boundary lines 60 are printed on the other outer side of the air bag 20 to indicate positions for the middle finger (C) of the rescuer.

In addition, boundary lines 60 are printed along the longitudinal direction of the air bag 20, so as to indicate positions for the index finger (B) of the rescuer in front of the positions for the middle finger (C), and positions for the ring finger (D) and little finger (E) of the rescuer in back of the positions of the middle finger (C).

In this case, when the air bag 20 is pressed by both hands of a rescuer, both hands of the rescuer can be located at positions spaced apart from the longitudinal mid-line 70 of the air bag 20.

However, the present invention is not limited to the above-described configurations. For example, the present invention can be applied to an air bag 20 for children. In this case, the distance between identifiers and the longitudinal mid-line 70 of the child air bag 20 may be determined based on the size of the child air bag 20.

Like the identifiers of the pressurization portions of the air bag 20 for right-handers, the identifiers of the pressurization portions of the air bag 20 for left-handers or ambidexters may include boundary lines 60, identification recesses, and an identification color(s) 62 (identification marks).

In addition, the BVM of the present invention may further include an alarming unit 90 for indicating time.

The alarming unit 90 may be disposed on the air bag or the connection hose. For example, the alarming unit 90 may be disposed on an end of the air bag as shown in FIG. 1.

In the present invention, the position of the alarming unit 90 is not limited to the above-mentioned positions. That is, the alarming unit 90 may be disposed at any position as long as the alarming unit 90 does not disturb the operation of pressing the air bag. In addition, the alarming unit 90 may be provided as an integral part or detachable part.

The alarming unit 90 produces an alarming sound as a discriminating sound every 6 seconds. In other words, the alarming unit 90 produces an alarming sound ten times regularly per minute.

For example, the alarming unit 90 may produce a basic sound once per second and a discriminating sound distinguishable from the basic sound every six seconds.

Therefore, when a rescuer presses the air bag, the rescuer can recognize time while listening to basic sounds and discriminating sounds so that he can continuously ventilate a patient with a constant, appropriate amount of air.

The alarming unit 90 is not limited thereto. For example, as well as the alarming unit 90 producing auditory discriminating sounds (alarming sounds), the alarming unit 90 may display visual alarming signals.

The alarming unit 90 may be electronically or mechanically configured by using any conventional parts.

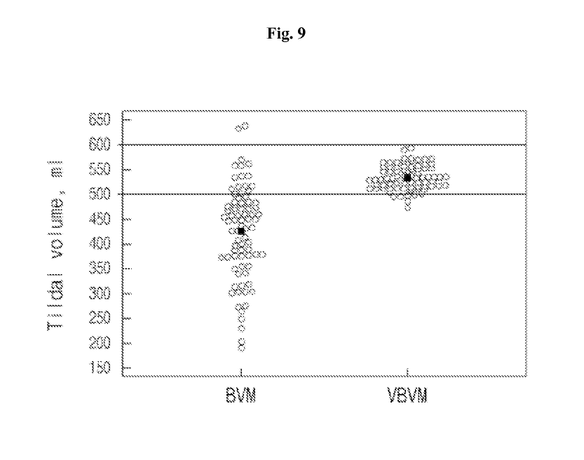

FIG. 9 is a graph showing the ventilation amounts of a BVM of the related art and the ventilation amounts of the BVM of FIG. 1 while the BVMs are used a plurality of times.

Referring to the drawing, the related-art BVM and the BVM of the present invention denoted by VBVM (volume-marked BVM) are compared based on a target ventilation amount (respiration amount) of air per pressing: 500 to 600 cc. According to the experimental results shown in the drawing, the ventilation amounts of air by the VBVM of the present invention are within the target ventilation amount range.

That is, the BVM of the present invention can be effectively used to continuously ventilate a patient with an appropriate ventilation amount of air: 500 to 600 cc.

While the present invention has been particularly shown and described with reference to the accompanying drawings according to exemplary embodiments, the present invention is not limited thereto. It will be understood by those of ordinary skill in the art that various changes in form and details may be made therein without departing from the spirit and scope of the present invention as defined by the following claims.

1. A bag valve mask (BVM) for appropriate ventilation, the BVM comprising pressurization portions formed on predetermined positions of an air bag as pressurization points which are pressed by tips of fingers of a rescuer when the rescuer holds the air bag with his fingers to artificially respirate a patient by continuously ventilating the patient with a constant amount of air.

2. The BVM of claim 1, comprising:
a mask configured to make contact with the noise and mouth of the patient in a state not allowing inflow of ambient air;
the air bag containing oxygen or air; and
a connection hose connecting the mask and the air bag,
wherein if the air bag is compressed, the oxygen or air contained in the air bag is supplied to the patient through the connection hose, the mask, and the respiratory tact of the patient,
wherein identifiers are formed on the air bag to indicate the pressurization portions.

3. The BVM of claim 2, wherein the identifiers are boundary lines printed as identification marks around regions of the air bag which are brought into contact with the tips of the fingers of the rescuer when the air bag is compressed by the rescuer.

4. The BVM of claim 3, wherein one of the boundary lines is printed on an outer side of the air bag at a position spaced 3 cm in a vertical direction from a center point on a longitudinal mid-line of the air bag so that a thumb of the rescuer is placed thereon, and another of the boundary lines is printed on the other outer side of the air bag at a position spaced 3 cm in the vertical direction from the center point so that a middle finger of the rescuer is placed thereon,
wherein other of the boundary lines are printed in a manner such that an index finger of the rescuer is located in front of the middle finger, and a ring finger and a little finger of the rescuer are located in back of the middle finger in a longitudinal direction of the air bag.

5. The BVM of claim 3, wherein one of the boundary lines is printed on the outer side of the air bag at a position spaced 3 cm in the other vertical direction from the center point on the longitudinal mid-line of the air bag so as to indicate a thumb position, and another of the boundary lines is printed on the other outer side of the air bag at a position spaced 3 cm in the other vertical direction from the center point so as to indicate a middle finger position,
wherein other of the boundary lines are printed so as to indicate an index finger position in front of the middle finger, and a ring finger position and a little finger position in back of the middle finger position along the longitudinal direction of the air bag.

6. The BVM of claim 3, wherein boundary lines are printed on the outer side of the air bag at positions spaced 5 cm in both vertical directions from the center point on the longitudinal mid-line of the air bag so as to indicate thumb positions, respectively, and boundary lines are printed on the other outer side of the air bag at positions spaced 5 cm in both the vertical directions from the center point so as to indicate middle finger positions,
wherein boundary lines are printed along the longitudinal direction of the air bag so as to indicate index finger positions in front of the positions for the middle finger positions, and ring finger positions and little finger positions in back of the middle finger positions.

7. The BVM of claim 4, wherein the boundary lines are rectangles having a side length in a range from 1.9 cm to 2.1 cm.

8. The BVM of claim 4, wherein the boundary lines are circles having a radius in a range from 0.9 cm to 1.1 cm.

9. The BVM of claim 4, wherein concave identification recesses are formed inside the boundary lines.

10. The BVM of claim 10, wherein the identification recesses are formed as a thumb identification recess and a middle finger identification recess on insides of the boundary lines on which a thumb and a middle finger of the rescuer are to be placed,
wherein when the air bag is compressed, inner surfaces of the air bag are brought into contact with each other at the thumb and middle finger identification recesses.

11. The BVM of claim 2, wherein the identifiers are concave identification recesses formed as identification shapes at positions of the air bag which make contact with the fingers of the rescuer when the air bag is compressed by the rescuer.

12. The BVM of claim 2, wherein the identifiers are an identification color(s) painted as identification marks on positions of the air bag which make contact with the fingers of the rescuer when the air bag is compressed by the rescuer.

13. The BVM of claim 2, wherein the identifiers comprise:
concave identification recesses formed at positions of the air bag which make contact with the fingers of the rescuer when the air bag is compressed by the rescuer; and
an identification color(s) painted on the identification recesses.

14. The BVM of claim 2, wherein the pressurization portions are formed at the predetermined positions of the air bag so that the patient is ventilated with 500 to 600 cc of oxygen or air when the air bag is compressed at the pressurization portions.

15. The BVM of claim 2, wherein the pressurization portions are formed at the predetermined positions of the air bag so that the patient is ventilated with 400 to 500 cc of oxygen or air when the air bag is compressed at the pressurization portions.

16. The BVM of claim 2, further comprising an oxygen or air cylinder disposed on the air bag.

17. The BVM of claim 2, wherein the air bag is formed of silicone or rubber.

18. The BVM of claim 2, wherein the connection hose comprises a one-way valve allowing oxygen or air to flow only in one direction from the air bag to the mask.

19. The BVM of claim 2, wherein an alarming unit is disposed on the air bag or the connection hose to provide information about time.

20. The BVM of claim 19, wherein the alarming unit produces a basic sound once per second and a discriminating sound distinguishable from the basic sound every six seconds.
